(19) United States
(12) Patent Application Publication
(10) Pub. No.: US 2026/0224287 A1
(43) Pub. Date: Aug. 6, 2026

(54) ROBOTIZED COMPUTER-ASSISTED SURGERY SYSTEM WITH PLANNING MODULE

(71) Applicant: ORTHOSOFT ULC, Montreal (CA)

(72) Inventors: Martin BRUMMUND, Montreal (CA); Anthony ROMANO, Columbia City, IN (US); Pierre COUTURE, Montreal (CA); Louise MUNRO, Montreal (CA); Louis-Philippe AMIOT, Montreal (CA); Walid CHAKER, Montreal (CA)

(21) Appl. No.: 19/529,784

(22) Filed: Feb. 4, 2026

Related U.S. Application Data
(60) Provisional application No. 63/754,078, filed on Feb. 5, 2025.

(51) Int. Cl. A61B 34/10; A61B 34/00; A61B 34/30

(52) U.S. Cl. CPC A61B 34/10; A61B 34/25; A61B 34/30; A61B 2034/105; A61B 2034/107; A61B 2034/256

(57) ABSTRACT

A computer-assisted surgery system may have a planning module for: obtaining a surgical procedure profile, the surgical procedure profile including a plurality of desired implanting parameters for at least one implant on a patient joint, the plurality of desired implanting parameters having parameter ranges defined by a minimum value and a maximum value, and at least one desired target output for the patient joint. Joint laxity data specific to the patient joint may be obtained. A surgical plan specific to the patient joint may be generated using the joint laxity data and joint anatomy, the surgical plan including selected implanting parameters within the parameters ranges to satisfy the desired target output(s). The surgical plan may be output to guide an implanting procedure for the patient joint.

### CROSS-REFERENCE TO RELATED APPLICATION

The present application claims the benefit of United States Patent Application No. 63/754,078 filed on February 5, 2025, the contents of which are incorporated herein by reference.

### TECHNICAL FIELD

The present disclosure pertains to computer-assisted surgery systems, and to robotic surgery systems, in which implants are implanted in patient joints, such as in computer-assisted orthopedic surgery.

### BACKGROUND OF THE ART

Computer-assisted surgery systems commonly provide an operator with navigation data during a surgical procedure. The navigation data may take various forms, including position and orientation data of tools relative to bones, predicted alterations, imaging, etc. The computer-assisted surgery systems may also include robotic apparatuses to assist a user in some bone alteration steps of surgical procedures.

Numerous factors and parameters are considered during a surgical procedure. For example, in knee arthroplasty, the positioning of an implant(s), such as a tibial component or the femoral component, results in bone removal, varus or valgus, joint laxity variations, and changes in knee rotation and flexion. Accordingly, in spite of computer-assisted surgery systems providing navigation data, there may nonetheless be some additional guidance provided to operators to further facilitate computer-assisted surgery for the outcome to be aligned with operator preferences and intentions.

### SUMMARY

In accordance with one aspect of the present disclosure, there is provided a computer-assisted surgery system comprising: a processing unit; and a non-transitory computer-readable memory communicatively coupled to the processing unit and comprising computer-readable program instructions executable by the processing unit for: obtaining a surgical procedure profile, the surgical procedure profile including a plurality of desired implanting parameters for at least one implant on a patient joint, the plurality of desired implanting parameters having parameter ranges defined by a minimum value and a maximum value, and at least one desired target output for the patient joint; obtaining joint laxity data specific to the patient joint; generating a surgical plan specific to the patient joint using the joint laxity data, the surgical plan including selected implanting parameters within the parameters ranges to satisfy the at least one desired target output; and outputting the surgical plan to guide an implanting procedure for the patient joint.

Further in accordance with the aspect, for instance, the computer-readable program instructions are executable by the processing unit for: obtaining the surgical procedure profile from a selection of a plurality of surgical procedure profiles.

Still further in accordance with the aspect, for instance, the computer-readable program instructions are executable by the processing unit for: providing the selection of the plurality of surgical procedure profiles specific to an operator of the computer-assisted surgery system.

Still further in accordance with the aspect, for instance, the computer-readable program instructions are executable by the processing unit for: providing the selection of the plurality of surgical procedure profiles each based on a condition of the patient joint.

Still further in accordance with the aspect, for instance, the computer-readable program instructions are executable by the processing unit for: providing the selection of the plurality of surgical procedure profiles each based on the condition of the patient joint, the condition being one of varus, valgus, neutral, and flexion contracture for a knee joint.

Still further in accordance with the aspect, for instance, the computer-readable program instructions are executable by the processing unit for: obtaining the surgical procedure profile in which the plurality of desired implanting parameters for a knee joint include one or more of: distal femoral resection, femur varus or valgus angle, flexion angle, posterior condylar axis angle, proximal tibial resection, tibial varus or valgus angle, posterior slope angle, posterior resection, and hip-knee-ankle angle.

Still further in accordance with the aspect, for instance, the computer-readable program instructions executable by the processing unit for: obtaining the surgical procedure profile in which the at least one desired target output for the patient joint includes a target range defined by a minimum value and a maximum value.

Still further in accordance with the aspect, for instance, the computer-readable program instructions are executable by the processing unit for: obtaining the surgical procedure profile in which the at least one desired target output for the patient joint includes a target value in addition to the target range defined by the minimum value and the maximum value, the target value being between by the minimum value and the maximum value.

Still further in accordance with the aspect, for instance, the computer-readable program instructions are executable by the processing unit for: obtaining the surgical procedure profile in which the at least one desired target output for the patient joint includes at least one of: residual gap in extension, residual gap in flexion, gap shape in extension, and gap shape in flexion.

Still further in accordance with the aspect, for instance, the computer-readable program instructions are executable by the processing unit for: obtaining a selection of implant from an operator prior to generating the surgical plan.

Still further in accordance with the aspect, for instance, the computer-readable program instructions are executable by the processing unit for: obtaining the joint laxity data specific to the patient joint being a knee joint, as one or more of: maximum flexion, maximum extension, varus/valgus value, medial and lateral gap values for an extension condition and a flexion condition, and total range of varus/valgus values.

Still further in accordance with the aspect, for instance, the computer-readable program instructions are executable by the processing unit for: obtaining the joint laxity data specific to the patient joint from a pre-operative assessment or an intra-operative assessment.

Still further in accordance with the aspect, for instance, the computer-readable program instructions are executable by the processing unit for: indicating when at least one of the desired implanting parameters is not within parameter ranges when generating the surgical plan specific to the patient joint.

Still further in accordance with the aspect, for instance, the computer-readable program instructions are executable by the processing unit for: prompting a user to accept and/or modify the at least one of the desired implanting parameters when not within the parameter ranges.

Still further in accordance with the aspect, for instance, the computer-readable program instructions are executable by the processing unit for: generating the surgical plan specific to the patient joint by including an identity of the implant and resection depths.

Still further in accordance with the aspect, for instance, the computer-readable program instructions are executable by the processing unit for: generating the surgical plan specific to the patient joint by including selected implanting parameters within the parameters ranges and a value for the at least one target output.

Still further in accordance with the aspect, for instance, the computer-readable program instructions are executable by the processing unit for: generating the surgical plan specific to the patient joint by including driving instructions for a robot.

Still further in accordance with the aspect, for instance, the robot is included, and the computer-readable program instructions are executable by the processing unit for: driving the robot in surgical workflow according to the driving instructions.

Still further in accordance with the aspect, for instance, a machine learning module is included and the computer-readable program instructions are executable by the processing unit to train the machine learning module using the surgical procedure profile, the joint laxity data and the surgical plan.

Still further in accordance with the aspect, for instance, the machine learning module is trained by accessing the surgical procedure profile, the joint laxity data and the surgical plan from computer-assisted surgery systems of prior surgical procedures.

Still further in accordance with the aspect, for instance, the machine learning module is trained by receiving patient data including one or more of age, gender, race, ethnicity, genetics, height, weight, body mass index, congenital conditions, pathologies, medical history.

Still further in accordance with the aspect, for instance, the machine learning module is trained by receiving post-operative assessment data associated with prior surgical procedures.

Still further in accordance with the aspect, for instance, the machine learning module is output as parametrized to generate surgical plans using surgical procedure profiles and joint laxity data.

### DESCRIPTION OF THE DRAWINGS

FIG. 1 is a schematic view of a robotized computer-assisted surgery (CAS) system with planning module in accordance with a variant of the present disclosure;

FIG. 2 is a block diagram of the robotized CAS system of FIG. 1, in accordance with a variant of the present disclosure;

FIGS. 2A-2F are example graphic-user interfaces (GUI) guiding a quantification of joint movement for the robotized CAS system of FIG. 1 and displaying varus and valgus angles of a knee in accordance with some embodiments;

FIG. 3 is an exemplary graphical user interface (GUI) setting out desired implanting parameters and target values as part of planning module of FIG. 2;

FIG. 4 is an exemplary GUI for selection of target values as part of planning module of FIG. 2;

FIG. 5 is an exemplary GUI for guiding an acquisition of landmarks in performing joint laxity assessment for the planning module of FIG. 2;

FIG. 6 is an exemplary GUI providing joint laxity data for the planning module of FIG. 2; and

FIG. 7 is an exemplary GUI displaying an output of surgical plan specific to a patient, by the planning module of FIG. 2.

### DETAILED DESCRIPTION

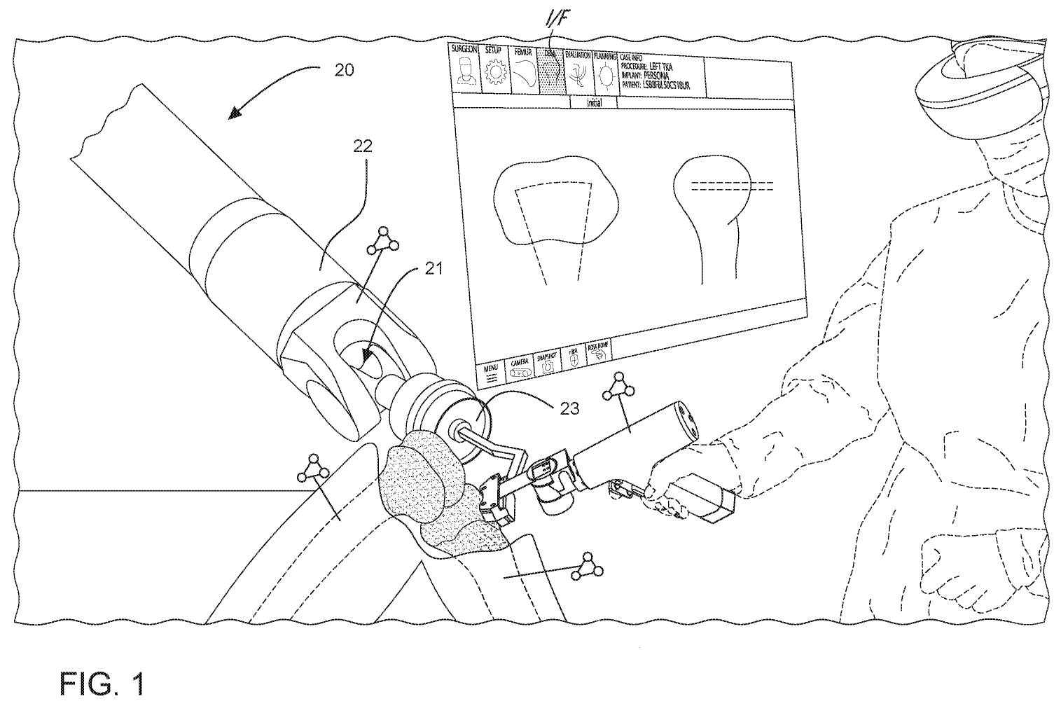

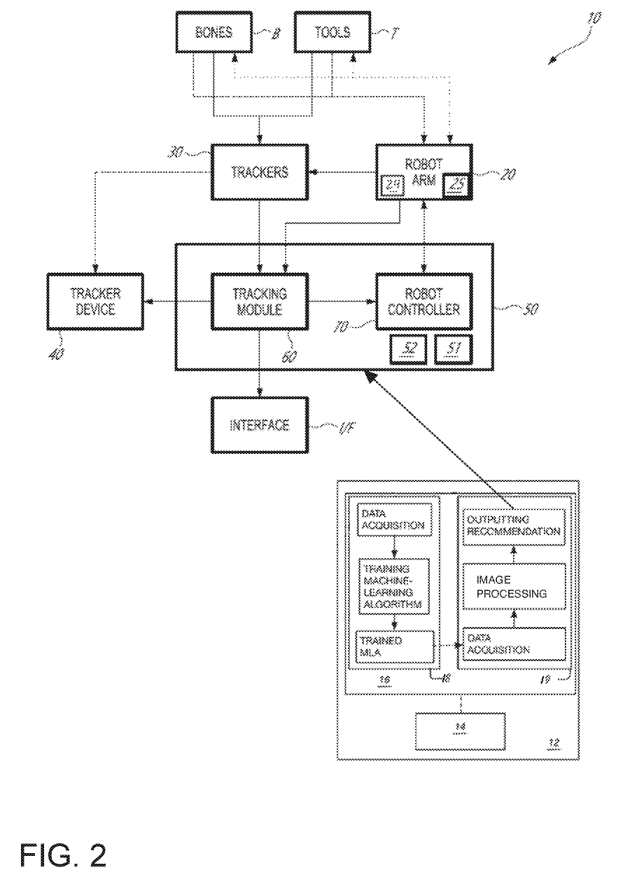

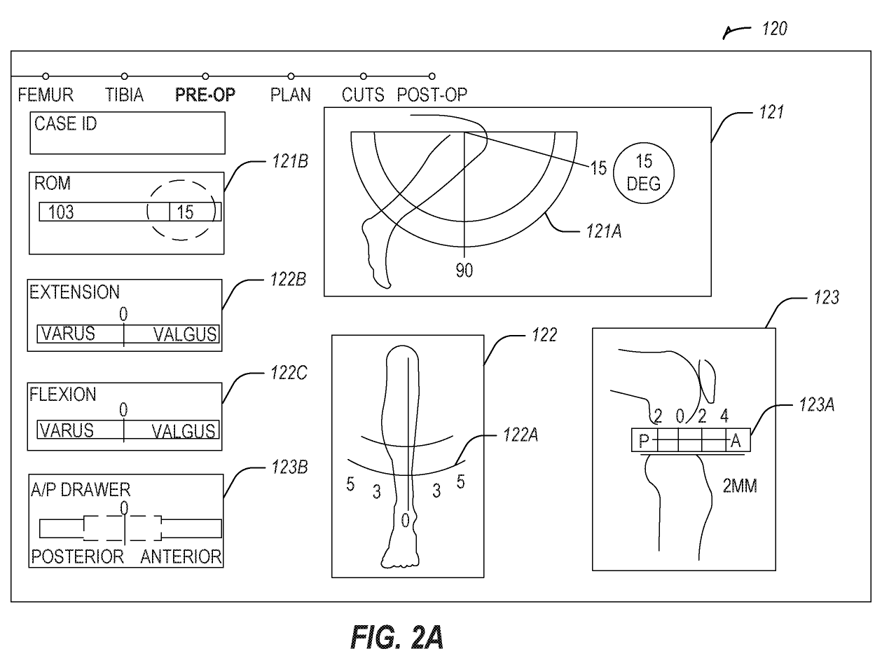

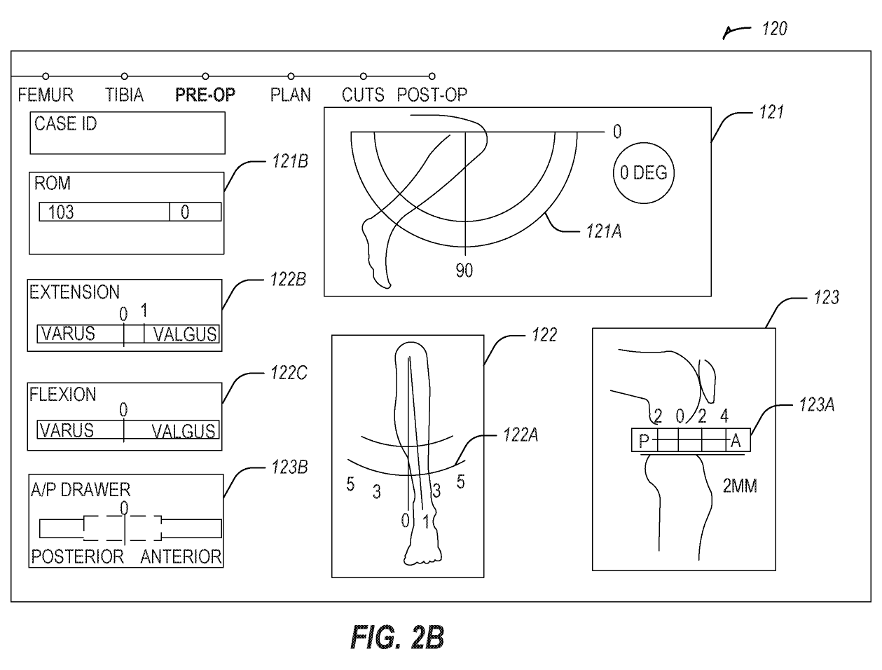

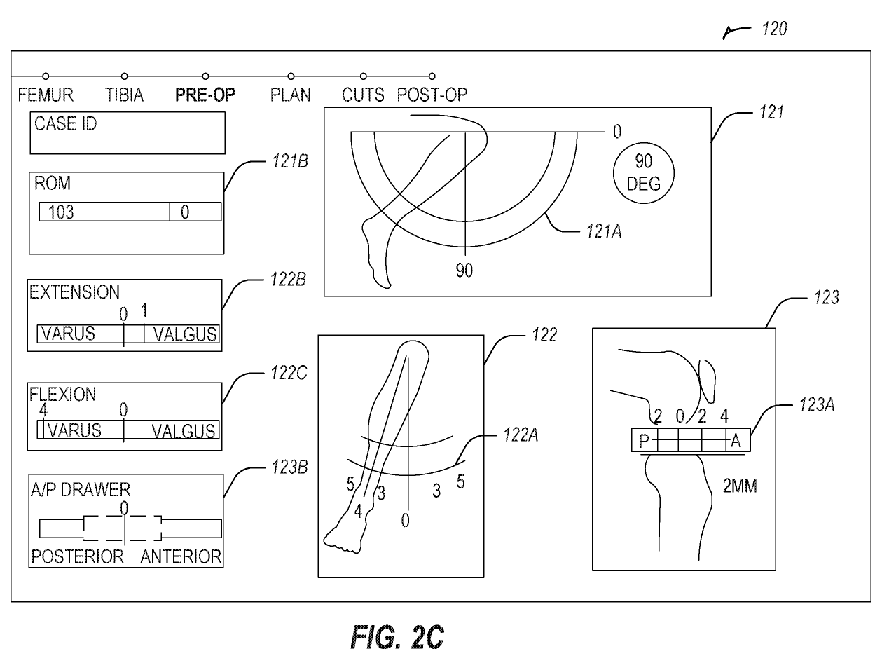

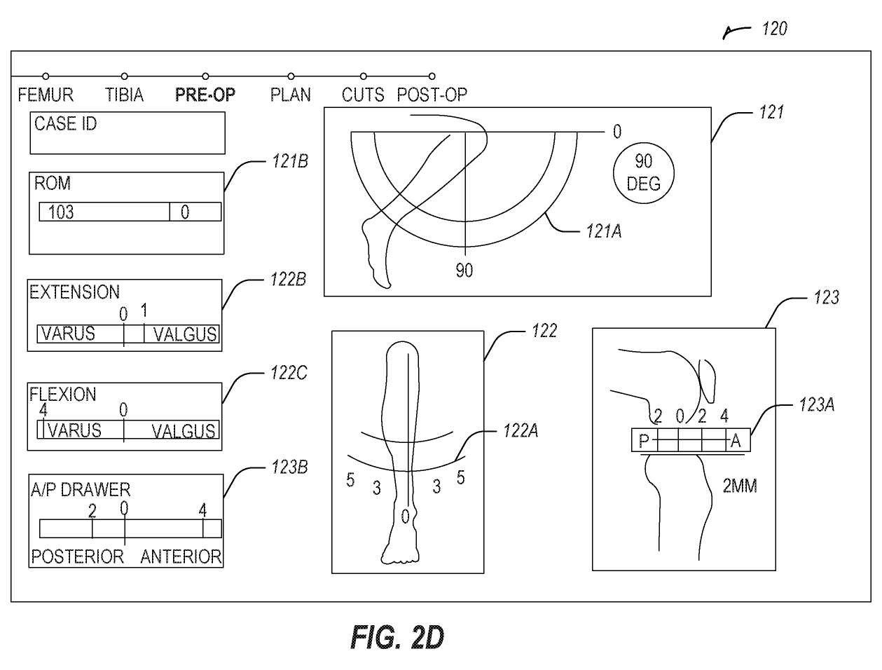

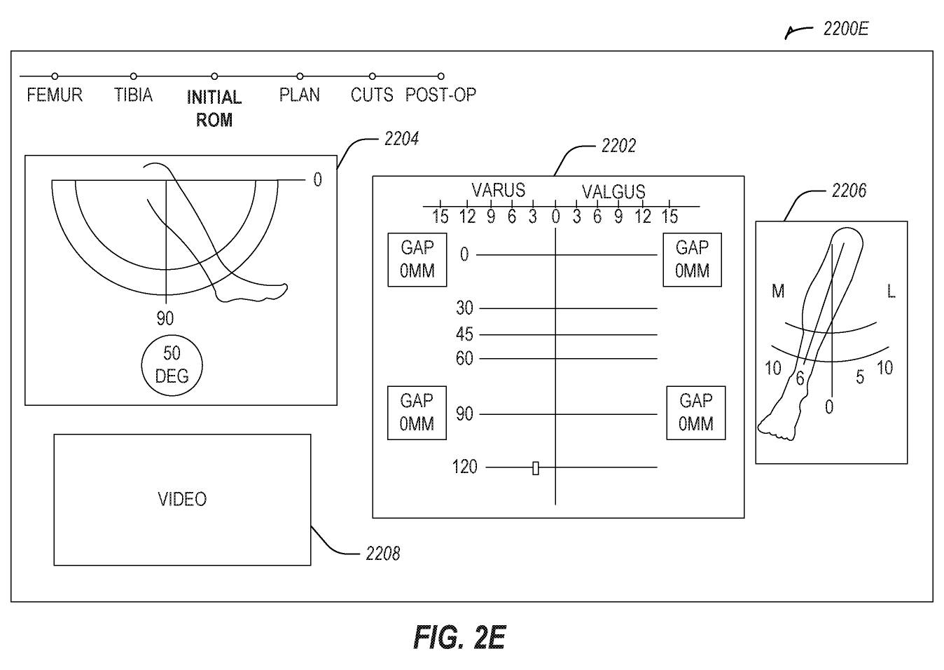

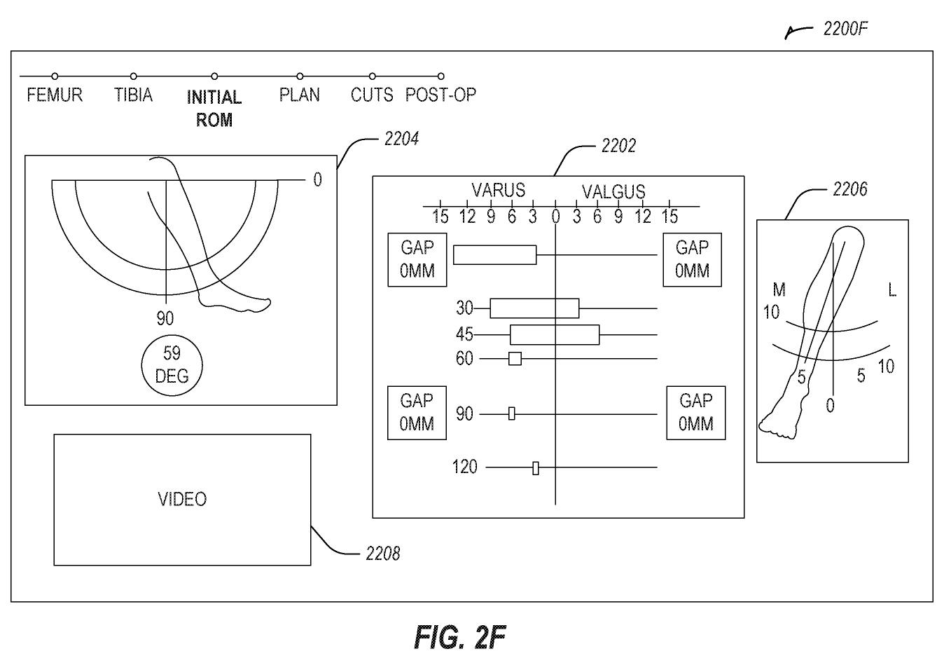

Referring to FIGS. 1 and 2, a computer-assisted surgery (CAS) system with planning module in accordance with a variant of the present disclosure. Referring to FIGS. 1 and 2, the CAS system 10 may optionally be robotized in a variant, and has, may have or may be used with a robot 20. The CAS system 10 may therefore be referred to as a robotized CAS system 10. For simplicity, the robotized CAS system may be referred to herein as the system 10 or CAS system 10. The CAS system 10 may optionally include a robot 20, with the present disclosure providing a description focusing on knee replacement, whether the knee replacement surgery involves a full or partial tibial plateau implant (a.k.a., tibial component) and/or a femoral implant (a.k.a., femoral component). The CAS system 10 could be used for other body parts, including non-exhaustively hip joint, spine, and shoulder bones, in orthopedic surgery in which bones are altered to receive implants, or other types of surgery, typically of the type in which joints are operated in, such as by the placement of an implant at the articulation.

Referring to FIGS. 1 and 2, the CAS system 10 has, may have or may be used with a robot 20, optical trackers 30, a tracker device 40, a CAS controller 50 (also known as a super controller 50), a tracking module 60, and a robot controller 70 (also known as a robot driver), an interface(s) I/F, or any combination thereof, with the CAS optionally receiving guidance from an assistance module 19 to guide its operations, the assistance module 19 described below.

Referring to FIG. 2, the CAS system 10 may optionally include or be in communication with a server 12 having one or more processing units 14, such as conventional central processing unit(s) (CPU(s)), and a non-transitory computer-readable memory 16 communicatively coupled to the one or more processing units 14. The memory 16 may store therein computer-readable instructions. The server 12 may be implemented in any suitable way. For example, in the present embodiment the server 12 is shown as a single entity, which may be a suitable computer for example with the associated communications hardware, input-output hardware, and controls hardware to give a few examples, all of which may be selected, for example from conventional hardware, to suit each particular embodiment of the other components of the CAS system 10. In other embodiments, the server 12 may be implemented in a distributed manner, with for example parts thereof being physical parts while other parts may be simulated, such as via virtual machines for example, and/or yet other parts thereof may be remote. The server 12 may also be cloud-based. A machine-learning module 18 is optionally present and part of the CAS system 10. The learning module of the CAS system 10 that receives data from surgical procedures to parametrize/train a machine-learning algorithm (MLA), which may be stored in the non-transitory memory 16 of the server 12, or in any other suitable non-transitory memory such as remote memory on the cloud, for example. The assistance module 19, optionally present in the CAS system 10, is the intervening module of the CAS system 10 that provides machine-learned assistance during a surgical procedure based on the parametrized/trained machine-learning algorithm (MLA).

The robot 20, shown by its robot arm in FIG. 1, may be present as the working end of the CAS system 10, and may be used to perform or guide bone alterations as planned by an operator and/or the CAS controller 50 and as controlled by the CAS controller 50. Bone alterations may include resecting bones to define resection planes for the subsequent placement of implants on the resection planes, drilling holes in the bones, and/or performing other maneuvers such as reaming the bone, etc.

While the robot arm 20 is displayed, the robot, robot system and robotic process may be implemented in the form of an orientation mechanism without a robotic arm. More specifically, interconnected links with joints (e.g., lockable joints) supporting a tool may constitute a robot in accordance with the present disclosure. The robot arm 20 may also be configured for collaborative/cooperative mode in which the operator may manipulate the robot arm 20. For example, the tooling end, also known as end effector, may be manipulated by the operator while supported and oriented by the robot arm 20, with the robot arm 20 holding its configuration when required, or moving along desired paths. The robot 20 may be the coordinate measuring machine (CMM) of the robotic system 10.

The optical trackers 30 are positioned on the robot 20, on patient tissue (e.g., bones B), and/or on the tool(s) T and surgical instruments, and provide tracking data for the robot 20, the patient and/or tools.

The tracking device 40, also known as a sensor device, apparatus, etc, performs optical tracking of the optical trackers 30, so as to enable the tracking in space (a.k.a., navigation) of the robot 20, the patient and/or tools.

The optical trackers 30 and tracking device 40 are an optional tracking modality of the robotic system 10. Other camera(s) may be present, for instance as a complementary registration tool or as a primary tracking tool. The camera may for instance be mounted on the robot 20, such as on the robot arm, such that the point of view of the camera is known in the frame of reference, also known as the coordinate system. Tracking may also be done using the robot 20 and its joint tracking capacity, as an alternative, or in addition to the tracking modalities described above. Moreover, other tracking modalities may include the use of inertial sensors, etc.

The CAS controller 50, also known as the super controller, includes the processor(s) and appropriate hardware and software to run a computer-assisted surgery procedure in accordance with one or more workflows. The CAS controller 50 may include or operate the tracking device 40, the tracking module 60, and/or the robot controller 70. As described hereinafter, the CAS controller 50 may also drive the robot arm 20 through a planned surgical procedure. The CAS controller 50 may be operated using a surgical workflow that may be based on the output of the planning module of the present disclosure.

The tracking module 60 is tasked with determining the position and/or orientation of the various relevant objects during the surgery procedure, such as the end effector of the robot arm 20, bone(s) B and tool(s) T, using data acquired by the tracking device 40 and by the robot 20, and/or obtained from the robot controller 70. The position and/or orientation may be used by the CAS controller 50 to control the robot arm 20.

The robot controller 70 is tasked with powering or controlling the various joints of the robot arm 20, based on operator demands or on surgery planning. The robot controller 70 may also optionally calculate robot movements of the robot arm 20, so as to control movements of the robot arm 20 autonomously in some instances, i.e., without intervention from the CAS controller 50. The robot controller 70 may be operated based on the control data provided by the CAS system 10 to operate a robotic computer-assisted surgery controller based on bone cut parameters associated with the implant parameters.

Other components, devices, systems, may be present, such as surgical instruments and tools T, interfaces I/F such as displays, screens, computer station, servers, and like etc. Secondary tracking systems may also be used for redundancy.

Referring to FIG. 1, if present, the robot arm 20 has a plurality of joints 21 and links 22, of any appropriate form, to support an end effector 23 that may interface with the patient, or may be used during surgery without interfacing with the patient. The end effector or tool head may optionally incorporate a force/torque sensor for collaborative/cooperative control mode, in which an operator manipulates the robot arm 20. The robot arm 20 is shown being a serial mechanism, arranged for the tool head 23 to be displaceable in a desired number of degrees of freedom (DOF). The tool head 23 may for example be a support that is not actuated, the support being used to support a tool, with the robot arm 20 used to position the tool relative to the patient. For example, the robot arm 20 controls 6-DOF movements of the tool head 23, i.e., X, Y, Z in the coordinate system, and pitch, roll and yaw. Fewer or additional DOFs may be present. The joints 21 are powered for the robot arm 20 to move as controlled by the CAS controller 50 (i.e., including the robot controller 70) in the six DOFs, and in such a way that the position and orientation of the end effector 23 in the coordinate system may be known, for instance by readings from encoders on the various joints 21. Therefore, the powering of the joints is such that the end effector 23 of the robot arm 20 may execute precise movements, such as moving along a single direction in one translation DOF, or being restricted to moving along a plane, among possibilities. Such robot arms 20 are known, for instance as described in United States Patent Application Serial no. 11/610,728, and incorporated herein by reference.

The end effector 23 of robot arm 20 may be defined by a chuck or like tool interface, typically actuatable in rotation. As a non-exhaustive example, numerous tools may be used as end effector for the robot arm 20, such tools including a registration pointer, a reamer (e.g., cylindrical, tapered), a reciprocating saw, a retractor, a camera, an ultrasound unit, a laser rangefinder or light-emitting device (e.g., the indicator device of US Patent No. 8,882,777), a laminar spreader, an instrument holder, or a cutting guide, depending on the nature of the surgery. The various tools may be part of a multi-mandible configuration or may be interchangeable, whether with human assistance, or as an automated process. The installation of a tool in the tool head may then require some calibration in order to track the installed tool in the X, Y, Z coordinate system of the robot arm 20.

The end effector 23 of the robot arm 20 may be positioned by the robot arm 20 relative to surgical area in a desired orientation according to a surgical plan, such as a plan based on preoperative imaging. In order to position the end effector 23 of the robot arm 20 relative to the patient B, the CAS controller 50 can manipulate the robot arm 20 automatically (without human intervention), or by a surgeon manually operating the robot arm 20 (e.g. physically manipulating, via a remote controller through the interface I/F) to move the end effector 23 of the robot arm 20 to the desired location, e.g., a location called for by a surgical plan to align an instrument relative to the anatomy. Once aligned, a step of a surgical procedure can be performed, such as by using the end effector 23.

The CAS system 10 may be used without the robot arm 20, with the operator performing manual tasks. In such a scenario, the CAS system 10 may only have the CAS controller 50, the tracking apparatus 60. Thus, the CAS system 10 may be used without robotic assistance, and may assist an operator by way of surgical navigation, i.e., tracking the surgical instrument(s) relative to the bone(s) in orthopedic surgery. When it operates the robot arm 20, the CAS system 10 may drive the robot arm 20 autonomously, and/or as an assistive or collaborative tool for an operator (e.g., surgeon). The surgical workflow of the CAS system may be operated based on the data from the planning module, as described below, to operate a robotic computer-assisted surgery controller based on bone cut parameters associated with the implant parameters.

Referring to FIG. 2, the trackers 30 may be secured to the bones B and at various locations on the robot 20, and may also or alternatively be on instruments. The trackers 30 may be known as trackable elements, markers, navigation markers, active sensors (e.g., wired or wireless) that may for example include infrared emitters. In a variant, the trackers 30 are passive retro-reflective elements, that reflect light. The trackers 30 have a known geometry so as to be recognizably through detection by the tracker device 40. For example, the trackers 30 may be retro-reflective lenses. Such trackers 30 may be hemispherical in shape, by way of a shield. The shield may be hollow and may cover a reflective membrane or surface. In an embodiment, the trackers 30 may be active emitters. In a variant, the trackers 30 may be NavitrackERs®.

The tracker device 40 may be embodied by an image capture device, capable of illuminating its environment. In a variant, the tracker device 40 may have two (or more) points of view, such that triangulation can be used to determine the position of the tracker devices 30 in space, i.e., the coordinate system of the CAS. The tracker device 40 may emit light, or use ambient light, to observe the trackers 30 from its points of view, so as to determine a position of the trackers 30 relative to itself. In an embodiment, the tracker device 40 is of the type known as the Polaris products by Northern Digital Inc. The tracker device 40 may form the complementary part of the CMM function of the CAS, with the trackers 30 on the robot base 20 for example.

Referring to FIG. 2, the CAS controller 50 is shown in greater detail relative to the other components of the robotic system 10. The CAS controller 50 has a processor unit 51 and a non-transitory computer-readable memory 52 communicatively coupled to the processing unit 51 and configured for executing computer-readable program instructions executable by the processing unit 51 to perform some functions, such as tracking the patient tissue and tools, using the position and orientation data from the robot 20 and the readings from the tracker device 40. The computer-readable program instructions may include the planning module of the present disclosure. The planning module may be accessed or computed by the processing unit 51 and may be stored in the computer-readable memory 52, and may be retrieved in cloud computing from any appropriate remote source.

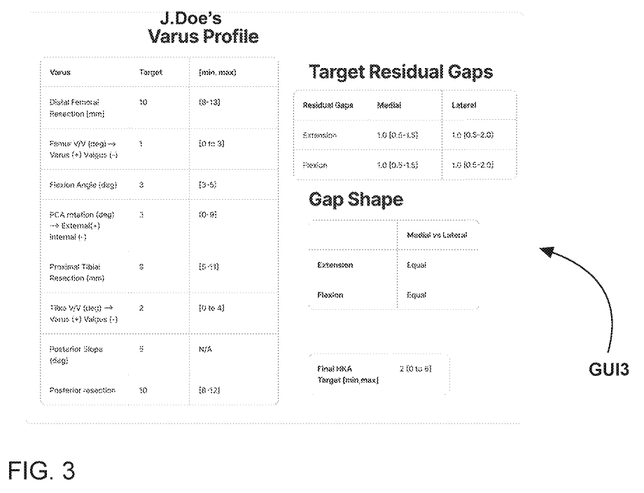

As part of the operation of the CAS controller 50, the computer-readable program instructions may include an operating system that may be viewed by a user or operator as a GUI on one or more of the interfaces I/F of the CAS system 10, with examples of the GUI being displayed starting with FIG. 3. Alternatively, some of the GUI displays provided herein may be accessed ahead of the surgical procedure as part of the planning, such that some of the GUI displays provided herein may not be viewed using the CAS system 10. It is via this or these interfaces that the user or operator may be guided by the planning module of the present disclosure, for example to interface with the planning module, to be guided by a surgical workflow, obtain navigation data, etc. The CAS controller 50 may also control the movement of the robot arm 20 via the robot controller module 70.

The CAS system 10 may include various types of interfaces I/F, for the information to be provided to the operator. The interfaces I/F may include and/or screens including wireless portable devices (e.g., phones, tablets), audio guidance, LED displays, head-mounted display for virtual reality, augmented reality, mixed reality, among many other possibilities. For example, the interface I/F comprises a graphic-user interface (GUI) operated by the CAS. The CAS controller 50 may also display images captured pre-operatively, or using cameras associated with the procedure (e.g., 3D camera, laparoscopic cameras, tool mounted cameras), for instance to be used in the collaborative/cooperative control mode of the system 10, or for visual supervision by the operator of the CAS, with augmented reality for example. The CAS controller 50 may drive the robot arm 20, in performing the surgical procedure based on the surgery planning achieved pre-operatively, or in maintaining a given position and orientation to support a tool. The CAS controller 50 may run various modules, in the form of algorithms, code, non-transient executable instructions, etc., in order to operate the CAS in the manner described herein. The CAS controller 50 may be part of any suitable processor unit, such as a personal computer or computers including laptops and desktops, tablets, server, etc.

The tracking module 60 may be a subpart of the CAS controller 50, or an independent module or system. The tracking module 60 receives the position and orientation data from the robot 20 and the readings from the tracker device 40. The tracking module 60 may hence determine the relative position of the objects relative to the robot arm 20 in a manner described below. The tracking module 60 may also be provided with models of the objects to be tracked. For example, the tracking module 60 may track bones and tools, and hence may use virtual bone models and tool models. The bone models may be acquired from pre-operative imaging (e.g., MRI, CT-scans), for example in 3D or in multiple 2D views, including with 2D X-ray to 3D bone model technologies. The virtual bone models may also include some image processing done preoperatively, for example to remove soft tissue or refine the surfaces that will be exposed and tracked. The virtual bone models may be of greater resolution at the parts of the bone that will be tracked during surgery, such as the knee articulation in knee surgery. The bone models may also carry additional orientation data, such as various axes (e.g., longitudinal axis, mechanical axis, etc.). The bone models may therefore be patient specific. It is also considered to obtain bone models from a bone model library, with the data obtained from the video images used to match a generated 3D surface of the bone with a bone from the bone atlas. The virtual tool models may be provided by the tool manufacturer, or may also be generated in any appropriate way so as to be a virtual 3D representation of the tool(s).

Additional data may also be available, such as tool orientation (e.g., axis data and geometry). By having access to bone and tool models, the tracking module 60 may obtain additional information, such as the axes related to bones or tools.

Still referring to FIG. 2, the CAS controller 50 may have the robot controller 70 integrated therein. However, the robot controller 70 may be physically separated from the CAS controller 50, for instance by being integrated into the robot 20 (e.g., in the robot base). The robot controller 70 is tasked with powering and/or controlling the various joints of the robot arm 20. The robot controller 70 may also optionally calculate robot movements of the robot arm 20, so as to control movements of the robot arm 20 autonomously in some instances, i.e., without intervention from the CAS controller 50. There may be some force feedback provided by the robot arm 20, for instance via the force sensor 24 or other sensors 25 to avoid damaging the bones, to avoid impacting other parts of the patient or equipment and/or personnel. The robot controller 70 may perform actions based on a surgery planning from the planning module.

Referring to FIGS. 2A-2F, a GUI 120 that may be provided by the CAS controller 50 is shown. The GUI 120 is used to guide the gathering of range-of-motion data of the tracked limbs, tracked in a coordinate system. In an embodiment, the GUI 120 guides a human operator, such as a surgeon or medical professional, in determining the limits of the range of motion and of joint laxity, based on force felt by the operator, as an alternative to using the force feedback capability of the robotized version of the system 10. According to FIG. 2A, a lateral leg display 121 may be provided to visually illustrate the limits of flexion and extension, with related angle. The operator manually displaces the tibia relative to the femur between maximum (flexion) and minimum (extension) angles, and the tracking of the tibia and femur by the tracking device 40 allows the processor to record these angles for use in a range-of-motion (ROM) analysis. The operator may assist in determining the maximum and minimum angle, by judging when to stop the extension and flexion based on the resistance felt. The leg display 121 may present the measured data in different forms, using for instance a movement arch 121A to visually show the range of movement. A ROM bar 121B may also be provided, showing the numerical values of angle, including a median angle. When the extension angle value is outside of standards, the ROM analysis may identify potential flexion contracture to influence the resection planning to remedy this issue. When the overall range of motion is below acceptable standards, the ROM analysis may identify this condition to influence resection planning and implant selection.

According to FIG. 2B, a frontal leg display 122 may also be provided in GUI 120 to visually illustrate the varus/valgus angles at extension and flexion. In a first step, the operator manually extends the leg, to then pivot the tibia relative to the femur to maximum varus and valgus angles, and the tracking of the tibia and femur by the tracking device 40 allows the ROM analysis to use these angles. The maximum varus/valgus angles may be determined by the operator’s judgement as to when to stop the extension and flexion based on the resistance felt. The frontal leg display 122 may provide the data in different forms, using also for example a movement arch 122A to visually show the range of movement, and an extension varus/valgus bar 122B, showing the numerical values of varus and valgus.

Then, according to FIG. 2C and using the same or another fontal leg display 122 and movement arch 122A, the operator manually flexes the leg, to then pivot the tibia relative to the femur to maximum varus and valgus angles, and the tracking of the tibia and femur allows the ROM analysis to use these angles. A flexion varus/valgus bar 122C may then show the numerical values of varus and valgus. These values are recorded for subsequent use by the processor in performing the soft tissue balancing. Moreover, these values may indicate a loose or tight knee condition, laterally or medially, whether it be correctable by implant positioning or not. In the latter case, the system 10 may suggest ligament releasing to remedy the condition. The soft tissue balancing may identify such a condition by being programmed with acceptable varus/valgus angle ranges. The varus/valgus angles obtained may be representative of the laxity of the medial and of the lateral collateral ligaments, as these ligaments delimit knee laxity. When the posterior and the anterior cruciate ligaments have not been resected (e.g., in a cruciate retaining surgery), these ligaments may also affect laxity. The knee articular capsule and the patellar tendon may also affect joint laxity.

Referring to FIG. 2D, an enlarged joint display 123 may also be provided to visually illustrate the anterior and posterior drawer distances at flexion. To gather the information, with the leg flexed, the operator manually pushes and pulls the tibia relative to the femur to maximum posterior and anterior positions, and the tracking of the tibia and femur by the tracking device 40 allows the ROM analysis to use the drawing positions, relative to a neutral position at which the tibia is natively positioned relative to the femur by soft tissue tension. Again, the maximum distances may be determined by the operator’s judgement as to when to stop the pushing and pulling based on the resistance felt. The joint display 123 may have different forms, using a distance scale 123A to visually show the range of movement, and a distance bar 123B, showing the numerical values of varus and valgus. These values are recorded for subsequent use during the soft tissue balancing. Joint displays 123A and 123B may also indicate a target laxity (for comparison) which is programmed to reflect the ideal laxity. The ideal laxity may be based on a surgeon-defined preference or suggested value from literature.

Therefore, at the outset of the surgical workflow steps guided by GUI 120, the system 10 has recorded joint laxity data, whether it be done preoperatively, perioperatively, intra-operatively and/or post-operativaly. The recorded information may be based on force feedback felt by the surgeon manipulating the tibia relative to the femur, or may be the result of manipulations by robotized components using sensors and output by the force measurement when the robotized components are programmed to limit force values. The recorded range of motion and joint laxity information may include maximum flexion angle, maximum extension angle, range of motion, varus and valgus angle values at extension, at flexion, or at any desired angle, anterior drawer distance, posterior drawer distance. The recorded information may be as a function of 3D bone models B of the tibia and femur, or of other bones in different surgical procedures. The order of information gathering using the GUI 120 may be changed from the order described above.

FIGS. 2E-2F illustrate graphical user interfaces (GUIs) 2200E and 2200F, which may be used for displaying flexion/extension angle, gaps, varus and valgus angles of a knee in accordance with some embodiments. The GUIs 2200E and 2200F include a video component 2208 to display real-time range of motion. The GUIs 2200E and 2200F include one or more graphical information components. For example, GUI 2200E shows the varus/valgus angle 2206 at 6 degrees varus in the medial direction at an flexion angle 2204 of 50 degrees (from full extension at 0 degrees). GUI 2200F shows the varus/valgus angle 2206 at 5 degrees varus in the medial direction at an flexion angle 2204 of 59 degrees (from full extension at 0 degrees). Additional information is shown at graphical information component 2202 in the GUIs 2200E and 2200F. The graphical information component 2202 includes gap information, varus/valgus angle information, range of motion information, and extension/flexion information. The range of motion information may be used to create a preoperative plan.

In an example, one or more of the GUIs 2200E or 2200F may provide a remote video or allow for a remote audio connection, such as with a remote surgeon. The remote video or remote audio may be a real-time connection to allow the remote surgeon to discuss a procedure or provide training with a local surgeon or to monitor the local surgeon. A GUI used by the remote surgeon may provide the remote surgeon with a video display of a surgical field operated by the local surgeon.

Referring to FIG. 3, the planning module may be used to provide a patient specific surgical plan (i.e., specifically defined for any given patient), according to the surgical planning preferences (e.g., in the form of parameters of surgery) of an operator such as a surgeon, a healthcare professional, as described below. The patient specific plan could also be based in literature and scientific articles, or generated by artificial intelligence. The surgical plan may include surgical workflow parameters that comply with selected implanting parameters, as detailed below, and may include a selection of implant(s), a geometry of the selected implant, including a 3D model of an implant (e.g., a virtual model), planned bone cuts, planned cut depths (e.g., planned resection depths and orientation). The selection of implant(s) may be a specific brand, and could be of a given size, but this is only an option as the selection of implant(s) may not be limited to a specific brand or size. The surgical plan may also have access to or generate information on joint alignment and/or gap balancing, such as performed as described with reference to FIGS. 2A-2F. The surgical workflow may include sequence or workflow of alterations to the bone (e.g., resection, drilling, etc.) with a sequence of surgical steps and tools used, navigation data for a user to perform the bone alterations, a robot driving file to operate the robot in assisting the healthcare professional in performing the surgical procedure in a collaborative mode, or to operate the robot in performing the surgical procedure, etc.

With reference to FIG. 3, an exemplary GUI display is shown at GUI3, in the context of knee surgery. The set up of GUI3 may differ, as may differ the information requested by the planning module. GUI3 may have an identity, herein labelled as “Dr. J. Doe’s Varus Profile” (i.e., Jane or John Doe). Therefore, GUI3 may be a profile for an individual named Dr. J. Doe, and may thus feature desired implanting parameters for Dr. J. Doe, i.e., implanting parameters preferences for Dr. J. Doe in his or her surgical planning. However, this is merely a way to call the profile. For example, a plurality of profiles may be recorded in the portal of or for a user named Dr. J. Doe, such that the operator’s name does not appear on GUI3, or appears in a different format. The profile may be referred to as a surgical procedure profile, and may be specific to a condition of the surgical procedure. For example, the profile may be named as a function of a condition, such as “extreme valgus”, “flexion contracture”, etc. In the example of GUI3 in FIG. 3, the condition of the surgical procedure is “Varus”, as the surgical procedure may attempt to replicate a native or current varus condition of the patient’s leg, i.e., a condition prior to the surgical procedure. As an example, Dr. J. Doe may also have a ”Valgus profile” and/or a “Neutral profile”, based on the pre-operative condition of the knee, or on a preference of Dr. J. Doe’s outcome.

The desired implanting parameters may be defined as including angles, dimensions, distances, that correlate one or more implants, the bone(s) on which the implant(s) is implanted, and the joint associated with the implant(s) and bone(s). The desired implanting parameters are not the actual implanting parameters, but are implanting parameters that comply with preferences or expected parameters for a given procedure. FIG. 3 shows desired implanting parameters for a knee, in the context of a total knee replacement surgery. Exemplary desired implanting parameters are shown in FIG. 3 as:

Distal Femoral Resection [mm]: the distal femoral resection relates to a distance value between the most distal point of the condyle(s) of the femur and a distal resection plane, upon which the femoral implant will lie. The units are listed as millimeters, but could be other units.

Femur V/V (deg): the femur V/V is the femoral varus or valgus angle value. For example, the angle value may be between a normal of the distal resection plane and a mechanical axis of the femur, projected on a frontal plane of the femur. Alternatively, the anatomical axis, femoral shaft axis or other axis could be used.

Flexion Angle (deg): the flexion angle is defined as the angle between the normal of the distal resection plane projected onto the sagittal plane and the mechanical axis of the femur. Other axes may be used, such as the angle between a femoral axis and a tibial axis with implants, when the knee is in full flexion. The femoral axis and the tibial axis may be the mechanical axes, the median axes, or other axes.

PCA rotation (deg): the posterior condylar axis angle (PCA) is the rotation of the femoral implant with respect to the posterior condylar axis during a total knee arthroplasty.

Proximal Tibial Resection (mm): the proximal tibial resection relates to a distance value between a point on the tibial plateau(s) and a proximal resection plane, upon which the tibial implant will lie. The units are listed as millimeters, but could be other units.

Tibia V/V (deg): the tibial V/V is the tibial varus or valgus angle value. For example, the angle value may be between a normal of the proximal resection plane of the tibia and a mechanical axis of the tibia, projected on a frontal plane of the tibia. Other axes could be used, such as the anatomical axis.

Posterior Slope: the posterior slope angle is defined as the angle between the normal of the tibial resection plane and the mechanical axis of the tibia projected onto the sagittal plane. Other axes could be used.

Posterior resection: the posterior resection relates to a distance value between a point on the posterior condyle(s) of the femur and a posterior resection plane, on which the femoral component will lie.

Final HKA: the HKA is the hip-knee-ankle angle, i.e., the angle measured between the mechanical axes of the femur and the tibia. The Final HKA could also be calculated as the sum of the planned varus/valgus on the femur and on the tibia.

There may be more or fewer desired implanting parameters, for example depending on the type of procedure. The implanting parameters may be entered preoperatively, for instance to give review time to the healthcare professional, to ensure that all surgical material is available. In some instances, it is possible for the healthcare professional to enter values intraoperatively.

In a variant, in the planning module, for each of the desired implanting parameters, the user must provide a target value or accept, but also a parameter range that includes a minimum value and a maximum value. It may not be necessary for all of the desired implanting parameters to have a target value, and a parameter range.

Still referring to FIG. 3, the profile may also include desired target outputs. The desired target outputs may be defined as the values that result from the implanting procedure, or that could be a projected value, such as a final HKA. Stated differently, the target outputs result from the various implanting parameters, i.e., how the implant is placed on the bone based on resections, etc. In the exemplary case of total knee replacement, both the femur and the tibia will have implants, such that the implanting parameters for both the tibia and the femur will have an impact on the target outcomes. In FIG. 3, the desired target values are:

Residual Gap in Extension: distance values are given for medial and lateral positions when the knee is in extension, and the distance values are between the femoral distal resection plane and the tibial resection plane, minus implant thickness, on the medial side and lateral side.

Residual Gap in Flexion: distance values are given for medial and lateral positions when the knee is in flexion, and the distance values are between the femoral posterior resection plane and the tibial resection plane, minus implant thickness, on the medial and lateral side.

Gap Shape in Extension: the gap shape explains a relationship between the residual gap values between a medial position and a lateral position, when the knee is in extension. The indication of equal means that the gap is the same in the medial position as in the lateral position, but other indications may include lateral<medial or lateral>medial.

Gap Shape in Flexion: the gap shape explains a relationship between the residual gap values between a medial position and a lateral position, when the knee is in flexion. The indication of equal means that the gap is the same in the medial position as in the lateral position, but other indications may include lateral<medial or lateral>medial.

There may be more or fewer desired implanting parameters, such as depending on the type of procedure.

In a variant, in the planning module, for at least some of the desired target output, the user must enter a target value, but also a target range that includes a minimum value and a maximum value. It may not be necessary for all of the desired target outputs to have a target value, and a target range. Additional information that can be input in GUI3 is the implant type (e.g., Persona CR, Persona CS, etc.). In such a case, during surgery, only the profiles applicable to the desired implant type will be considered in the planning panel.

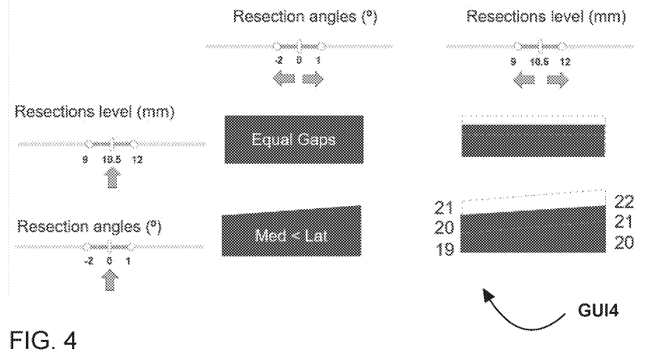

Referring to FIG. 4, there is illustrated another exemplary display GUI, shown as GUI4, that may be part of the planning module, used in preoperative planning. GUI4 may have various scales to set the target values, such as for resection angle (in degrees) and for resection level (in mm or other distance value). GUI4 provides an illustration of gap shape as a function of resection angles, to facilitate selection by the healthcare professional. This is a possible representation allowing the surgeon, or user, to create preference profiles to guide the smart planning module. Each profile may contain, without being limited to, criteria for resection depths and angles (respectively identified as “resection level (mm)” and “resection angles (º)” for given bones (i.e. the femur and the tibia). Each of these parameters may be defined by, without being limited to, a target value as well as a minimum and maximum acceptable range. The other diagrams in the figure reference the total space of the compartments, either medial or lateral (in flexion and in extension) as well as the shape criteria to target when balancing the knee. I.e. equal gaps between the medial and lateral compartments, or a specific laterally tightness (i.e. medial < lateral).

The planning module may thus produce a patient-specific surgical plan, in which the surgical solution (i.e., the selected implanting parameters) will not exceed the parameter ranges set by the minimum value and the maximum value set by the desired implanting parameters. Stated differently, the user profile will include the desired implanting parameters with parameter ranges, and the surgical plan will include selecting implanting parameters that are within the parameter ranges. The surgical plan may find the closest solution if no solution satisfies parameter ranges. Alternatively, the surgical plan may indicate when one or more desired implanting parameters is(are) not within parameter ranges, and may prompt a user to accept and/or modify the desired implanting parameter(s).

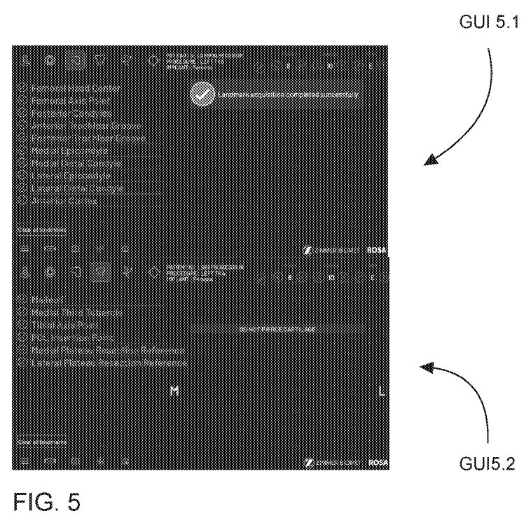

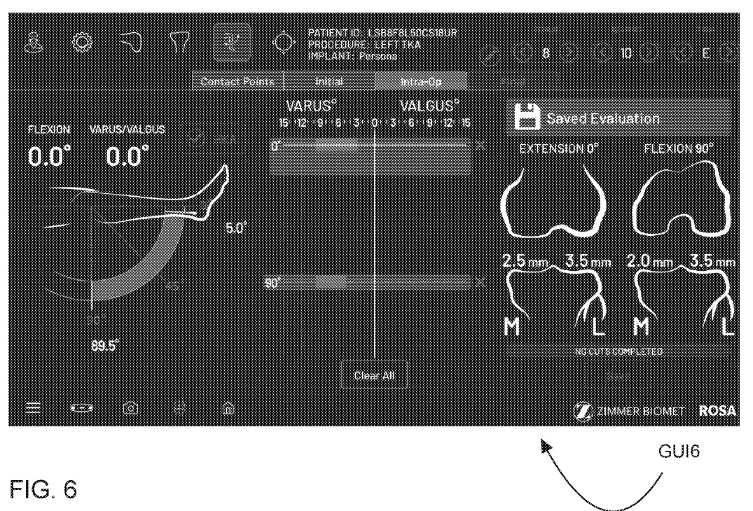

The surgical plan may depend on joint laxity data for the knee, that may be obtained in different ways. As observed from FIG. 6, the joint laxity data may be obtained intraoperatively, when the distal femur, proximal tibia, ligaments and other soft tissues are exposed. The joint laxity data may also rely on manipulations made by a user and/or by a robot for joint laxity values to be recorded, as explained above for FIGS. 2A-2F. In a variant, it may therefore be necessary to register the bones in the referential system of the CAS system 10. In a variant, trackers 30 may be positioned on the bones, such as on the femur and the tibia in the example of a knee surgery. It may be required to digitize some points on the surfaces of the bones, and FIG. 5 provides an example of a display GUI5.1 identifying a series of landmarks that can be acquired, such as digitized (e.g., posterior condyles), while other landmarks are the result of manipulations (e.g., femoral head center). GUI5.2 is for the tibia. As observed in GUI5.1 and in GUI5.2, an indicator (e.g., a check mark) can confirm that a landmark has been registered in the coordination system.

Referring to FIG. 6, the joint laxity data may include the maximum flexion, and the maximum extension. Joint laxity data may also include a varus/valgus value, such as for the current hip-knee-ankle angle, at maximum flexion, at maximum extension. The joint laxity data may also provide a total range of varus/valgus values. This may be shown by the scale in GUI6. Moreover, medial and lateral gap values may be provided for an extension condition and a flexion condition, as shown by the pictograms in GUI6.

The joint laxity data may also come from a pre-operative evaluation. The pre-operative evaluation may be done from a manual assessment by an operator or by a robot. Alternatively, the joint laxity may be obtained from a surgical assistance system for joint laxity assessment as described in PCT Patent Application No. WO2026/000072 , incorporated herein by reference. Such surgical assistance system for joint laxity assessment may provide a joint laxity assessment using available images (e.g., radiographic images) and may rely on a machine-learning module to provide an assessment.

Therefore, using a surgical procedure profile, such as Dr. J. Doe’s Varus Profile of FIG. 3, as well as joint laxity data that may be obtained according to different approaches, the planning module may generate a surgical plan specific to the patient joint. The surgical plan may include selected implanting parameters within the parameters ranges set as part of the Profile of FIG. 3. Moreover, the surgical plan aims to satisfy the desired target output(s) also defined as part of the Profile of FIG. 3.

In a variant, the surgical plan is generated using an appropriate algorithm. One possible solution is the revisited Depth-First Search* (DFS) where each node represents a value that a variable (resection values, VV, etc.) can take. The planning module iterates by decreasing/increasing the values to find the best combination that minimize one or more cost functions. As an approach, all desired implanting parameters may be set to their target values. A first search may be performed to satisfy the desired shape by mainly varying tibia and femur VVs, PCA rotation for example. As a possibility, the PCA slope could also be modified. If the shape step succeeds, a second search may be performed by varying the resection depths to satisfy the target gap values. If multiple solutions are possible, the planning module may select implanting parameters to optimize target resection and angle ranges to be closest to target values, while prioritizing tighter gaps as target outputs. The algorithm will also automatically select the best femoral component size that satisfies the search criteria and could also be applied to the tibial implant and the bearing thickness.

The surgical plan may be said to be patient specific, in that it may include a selection of profile (e.g., FIG. 3) based on patient assessment. Moreover, the surgical plan is based on the joint laxity data for the patient.

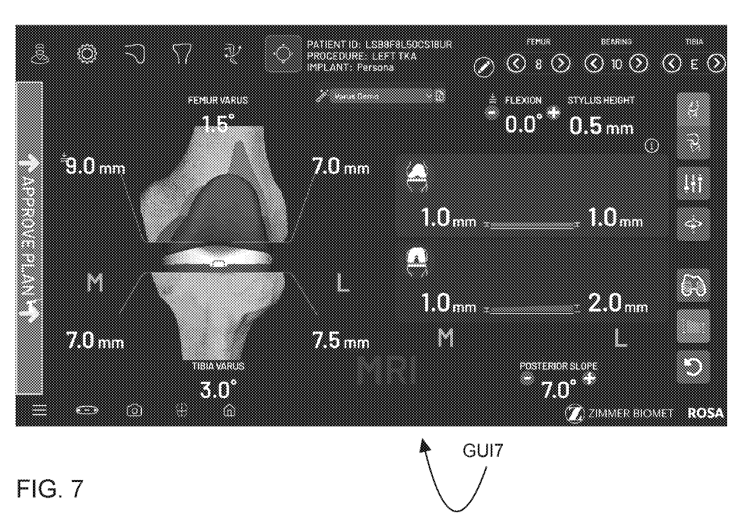

Referring to FIG. 7, the patient-specific surgical plan may include all necessary information to execute the surgical procedure, and may include the type of implant (e.g., brand, model, dimensions), the model of the bone with planned resection. In a variant, the patient-specific surgical plan provides its output in the form of GUI7. The surgical plan of FIG. 7 is specific to the patient joint as the joint laxity data was used in generating the surgical plane and resection levels and orientations are based on the landmarks acquired during the surgery. The surgical plan includes selected implanting parameters within the parameters ranges to satisfy the desired target output(s). Thus, the surgical plan exemplified in GUI7 may include selected implanting parameters such as resection depths, shown as being provided in both medial and lateral values, for the femur and the tibia, in the scenario of total knee replacement. Other selected implanting parameters shown in GUI7 include the femur V/V, depicted as a Valgus in FIG. 7, the tibia V/V, illustrated as a tibia varus, the posterior slope, the flexion angle, among others. The selected implanting parameters correspond to the desired implanting parameters of FIG. 3, and are within the parameter ranges, as per the simulations performed by the planning module. The surgical plan may also include target outputs such as the residual gap values that correspond to the target outputs of the profile of FIG. 3. In GUI7, the target outputs are shown as pictograms and values, again being provided in both medial and lateral values, for the femur and the tibia, in the scenario of total knee replacement. GUI7 could also show the predicted final HKA, i.e., one of the parameters of FIG. 3.

If the CAS system 10 is robotized, the patient-specific planning file may include driving instructions for the robot arm 20 to position itself for the resection to be performed based on the patient-specific planning file. If the CAS system 10 is not robotized, the patient-specific planning file may include navigation instructions relative to the bone for an operator to perform bone alterations based on the planning.

Therefore, the planning module may be described as obtaining a surgical procedure profile, such as among numerous procedure profiles, that may be from a given healthcare professional, and/or representative of different anatomical conditions. The surgical procedure profile may include a plurality of desired implanting parameters for one or more implants on a patient joint. The desired implanting parameters may be defined as including angles, dimensions, distances, that correlate one or more implants, the bone(s) on which the implant(s) is implanted, and the joint associated with the implant(s) and bone(s). The desired implanting parameters are not the actual implanting parameters, but are implanting parameters that comply with preferences or expected parameters for a given procedure. FIG. 3 provides exemplary desired implanting parameters, for knee replacement surgery. The plurality of desired implanting parameters have parameter ranges defined by a minimum value and a maximum value. The desired implanting parameters may each also include a target value that is within the parameter ranges. The surgical procedure profile may also include one or more desired target outputs for the patient joint after implanting. The desired target outputs may be defined as the values that result from the implanting procedure. FIG. 3 provides exemplary desired target outputs, for knee replacement surgery. In some cases, the desired target outputs may include a target value and a target range.

The planning module may obtain joint laxity data specific to the patient joint. In a variant, the joint laxity data is obtained by performing maneuvers during the surgical procedure, for example using FIGS. 5 and 6 as an example. The joint laxity data may require that the bones define the joint, i.e., femur and tibia for knee replacement surgery, are registered for tracking in a coordinate system of the CAS system 10. The maneuvers may then be performed by the healthcare professional, with or without robotic assistance. FIG. 6 exemplifies joint laxity data that may be obtained. In a variant, such maneuvers are performed pre-operatively, such that the planning module obtains a joint laxity data file specific to a patient, which file may result from a pre-operative assessment. As another option given above, the pre-operative assessment may be done by image processing, using a machine-learning module.

Using the joint laxity data and anatomical landmarks or joint anatomy/anatomical structures, the planning module may therefore generate a surgical plan specific to the patient joint. The surgical plan may include selected implanting parameters within the parameters ranges defined for example in the surgical procedure profile, to satisfy one or more desired target outputs of FIG. 3. The surgical plan may be output to guide an implanting procedure for the patient joint. An example of the output format of the surgical plan is given in FIG. 7. Other formats may be in the form of navigation instructions, robot driving instructions. Moreover, the surgical plan may include other data, such as an implant selection (i.e., brand, model and/or size).

The procedure set out above to generate a surgical plan specific to a patient may generally be described as obtaining a surgical procedure profile, the surgical procedure profile including a plurality of desired implanting parameters for at least one implant on a patient joint, the plurality of desired implanting parameters having parameter ranges defined by a minimum value and a maximum value, and at least one desired target output for the patient joint after implanting; obtaining joint laxity data specific to the patient joint; generating a surgical plan specific to the patient joint using the joint laxity data, the surgical plan including selected implanting parameters within the parameters ranges to satisfy the at least one desired target output; and outputting the surgical plan to guide an implanting procedure for the patient joint.

When the expressions “patient specific” and “specific to a patient” are used, it is meant that the data, the surgical plan, the output are associated with one particular patient, based for example on that specific patient’s anatomy, condition, expectations. The surgical plan and the output may not be used on other patients without due verification, and any correspondence between patients may be fortuitous. It is also possible for the system to provide default profiles that would fit typical pathologies. Such «default profiles» could be transformed as «template profiles» needing to be reviewed/adapted by the surgeon.

Referring to FIG. 2, the ML module 18 performs data acquisition for subsequent training of the MLA. The MLA may be trained to perform some of the tasks of the planning module, including generating a patient-specific surgical plan according to the surgical procedure profile (e.g., selected by the healthcare professional), using the joint laxity data. After data acquisition, the ML module 18 trains its machine-learning algorithm (MLA). Procedural data of the surgical procedures may be provided to the ML module 18. The visual cues or aspects may also be correlated to the surgical plan. Moreover, post-operative assessments may also be considered, to further train the ML module 18. Additional information may be used for the training, such as data on the implant that was selected for the patient. The implant that was selected may be in the form of a brand, a type, a model, a serial number, and may also include information such as dimensions, a three-dimensional model, elevation views, etc.

The data acquisition may take other forms as well. The ML module 18 may supplement surgical plan and implant type with patient-related data, such as non-confidential data for privacy reasons. The data that is part of the patient file obtained by the ML module 18 may include patient data such as age, gender, race, ethnicity, genetics, height, weight, body mass index, congenital conditions, pathologies, medical history, etc. The data acquired by the ML module 18 may also include surgical flow information from the past surgical procedures, as well as control data from a CAS system, such as from the CAS controller 50 (FIG. 2), that performed the past surgical procedures. This may include an identification of tools used, bones being altered, navigation data (e.g., position and/or orientation of tools relative to the bones in a referential system) the parameters of alteration (depth of cuts, orientation, navigation data), navigation of robot arm 20 if present.

In some instances, an assessment of the surgery is done post-operatively, and may be added to the patient file. The ML module 18 may access this information as part of data acquisition as well. The assessment of surgery may take various forms, including quantitative data. In the case of orthopedic surgery, the quantitative data may be joint laxity values, distance or length data, such as limb length discrepancy, resection depths. The quantitative data may be orientation data, such as varus/valgus, offset, tilt, etc. The quantitative data may be volumetric data, such as volume of bone removed, volume of resection. The assessment may also include qualitative data, with patient feedback including pain level, perceived mobility, joint looseness, patient satisfaction score, etc. The assessment data may be acquired over a rehabilitation period, with post-operative patient follow ups and the use of wearable sensor technologies, for instance over an extended period of time.

The training of the ML algorithm may be based on training data acquired from multiple prior surgeries, in different locations, from different CAS systems 10, and/or involving different surgeons. The training of the ML algorithm in the ML module 18 may include at least 100 surgical procedures, without an upper echelon of review. The machine learning algorithm may be trained with or without supervision by image processing radiographic images of surgeries for patients of different age, gender, race, ethnicity, genetics, height, weight, body mass index, congenital conditions, pathologies, medical history, etc., to train the ML algorithm with procedures covering a wide diversity of cases, including standard cases, and deviation cases. Age, gender, race, ethnicity, genetics, height, weight, body mass index, congenital conditions, pathologies, medical history, etc. may have an impact on the type of implant selected in prior surgical procedures used as training data for the ML module 18. Training can also be done based on surgeon preferences.

As a consequence of the training of the ML algorithm, the learning module 18 may produce and output a parametrized ML algorithm. The ML algorithm may be selected from different supervised machine learning algorithms, such as neural networks, Bayesian networks, support vector machines, instance-based learning, decision trees, random forests, linear classifiers, quadratic classifiers, linear regression, logistic regression, k-nearest neighbor, hidden Markov models, or the like. The ML algorithm may be selected from different unsupervised machine learning algorithms, such as expectation-maximization algorithms, vector quantization, and information bottleneck method.

Thus, if available, the assessment of the surgery done post-operatively, such as in the form of the quantitative data and/or qualitative data, may be associated with the surgical plan to train the ML algorithm in evaluating the success of a surgical procedure, and its numerous parameters as a function of post-operative assessment. There results a trained ML algorithm in the ML module 18. The trained ML algorithm may have the capacity of performing various functions through its training, including outputting a surgical plan from joint laxity data and a surgical procedure profile (e.g., FIG. 3). As part of the surgical plan, the trained ML algorithm may provide implant recommendations.

Referring to FIG. 2, the assistance module 19 has been updated with the parametrized machine learning algorithm. The assistance module 19 may be the part of the CAS system 10 that is used pre-operatively, peri-operatively, or intraoperatively, to assist the planning module in generating a surgical plan. The surgical plan may include a recommended implant type. In an embodiment, the assistance module 19 accesses the server 12 in which the parametrized ML algorithm is located, for instance locally in edge computing, but in other embodiments the ML algorithm may be on the cloud for example. Pre-operatively, peri-operatively, and/or intraoperatively, data acquisition is performed by the assistance module 19, to output surgical plan data to the planning module.

Other forms of data acquisition may be used by the assistance module 19. This may also include accessing the patient files, with its content that may be as described above. For example, this may include patient data such as age, gender, race, ethnicity, genetics, height, weight, body mass index, congenital conditions, pathologies, medical history, etc.

Based on this, the assistance module 19 may output the surgical plan or data used as part of the surgical plan generated by the planning module. Optionally, part of the surgical plan is optionally provided to a CAS controller, such as that shown at 50 in FIG. 2. If the CAS system is of the type having a robot, such as robot arm 20 in FIG. 1, the assistance module 19 may output and provide control data to operate the robotic computer-assisted surgery controller based on the surgical plan specific to the patient. Based on output from the parametrized machine learning algorithm, the assistance module 19 may propose a surgical procedure flow using the available data. There may result improvements in the assistance provided by the system 10, for instance in providing a solution based on other procedures and post-operative assessment. Moreover, with the guidance provided by the assistance module 19 may cause a gain of efficiencies in the surgical procedure, lessening computing requirements in the operating room for the system 10.

What is claimed is:

1. A computer-assisted surgery system comprising:
a processing unit; and
a non-transitory computer-readable memory communicatively coupled to the processing unit and comprising computer-readable program instructions executable by the processing unit for:
obtaining a surgical procedure profile, the surgical procedure profile including
a plurality of desired implanting parameters for at least one implant on a patient joint, the plurality of desired implanting parameters having parameter ranges defined by a minimum value and a maximum value, and
at least one desired target output for the patient joint;
obtaining joint laxity data specific to the patient joint;
generating a surgical plan specific to the patient joint using the joint laxity data, the surgical plan including selected implanting parameters within the parameters ranges to satisfy the at least one desired target output; and
outputting the surgical plan to guide an implanting procedure for the patient joint.

2. The computer-assisted surgery system according to claim 1, wherein the computer-readable program instructions are executable by the processing unit for: obtaining the surgical procedure profile from a selection of a plurality of surgical procedure profiles.

3. The computer-assisted surgery system according to claim 2, wherein the computer-readable program instructions are executable by the processing unit for: providing the selection of the plurality of surgical procedure profiles specific to an operator of the computer-assisted surgery system.

4. The computer-assisted surgery system according to claim 2, wherein the computer-readable program instructions are executable by the processing unit for: providing the selection of the plurality of surgical procedure profiles each based on a condition of the patient joint.

5. The computer-assisted surgery system according to claim 1, wherein the computer-readable program instructions are executable by the processing unit for: providing the selection of the plurality of surgical procedure profiles each based on the condition of the patient joint, the condition being one of varus, valgus, neutral, and flexion contracture for a knee joint.

6. The computer-assisted surgery system according to claim 1, wherein the computer-readable program instructions are executable by the processing unit for: obtaining the surgical procedure profile in which the plurality of desired implanting parameters for a knee joint include one or more of: distal femoral resection, femur varus or valgus angle, flexion angle, posterior condylar axis angle, proximal tibial resection, tibial varus or valgus angle, posterior slope angle, posterior resection, and hip-knee-ankle angle.

7. The computer-assisted surgery system according to claim 1, wherein the computer-readable program instructions executable by the processing unit for: obtaining the surgical procedure profile in which the at least one desired target output for the patient joint includes a target range defined by a minimum value and a maximum value.

8. The computer-assisted surgery system according to claim 7, wherein the computer-readable program instructions are executable by the processing unit for: obtaining the surgical procedure profile in which the at least one desired target output for the patient joint includes a target value in addition to the target range defined by the minimum value and the maximum value, the target value being between by the minimum value and the maximum value.

9. The computer-assisted surgery system according to claim 1, wherein the computer-readable program instructions are executable by the processing unit for: obtaining the surgical procedure profile in which the at least one desired target output for the patient joint includes at least one of: residual gap in extension, residual gap in flexion, gap shape in extension, and gap shape in flexion.

10. The computer-assisted surgery system according to claim 1, wherein the computer-readable program instructions are executable by the processing unit for: obtaining a selection of implant from an operator prior to generating the surgical plan.

11. The computer-assisted surgery system according to claim 1, wherein the computer-readable program instructions are executable by the processing unit for: obtaining the joint laxity data specific to the patient joint being a knee joint, as one or more of: maximum flexion, maximum extension, varus/valgus value, medial and lateral gap values for an extension condition and a flexion condition, and total range of varus/valgus values.

12. The computer-assisted surgery system according to claim 1, wherein the computer-readable program instructions are executable by the processing unit for: obtaining the joint laxity data specific to the patient joint from a pre-operative assessment or an intra-operative assessment.

13. The computer-assisted surgery system according to claim 1, wherein the computer-readable program instructions are executable by the processing unit for: indicating when at least one of the desired implanting parameters is not within parameter ranges when generating the surgical plan specific to the patient joint.

14. The computer-assisted surgery system according to claim 13, wherein the computer-readable program instructions are executable by the processing unit for: prompting a user to accept and/or modify the at least one of the desired implanting parameters when not within the parameter ranges.

15. The computer-assisted surgery system according to claim 1, wherein the computer-readable program instructions are executable by the processing unit for: generating the surgical plan specific to the patient joint by including an identity of the implant and resection depths.

16. The computer-assisted surgery system according to claim 15, wherein the computer-readable program instructions are executable by the processing unit for: generating the surgical plan specific to the patient joint by including selected implanting parameters within the parameters ranges and a value for the at least one target output.

17. The computer-assisted surgery system according to claim 1, wherein the computer-readable program instructions are executable by the processing unit for: generating the surgical plan specific to the patient joint by including driving instructions for a robot.

18. The computer-assisted surgery system according to claim 17, including the robot, and wherein the computer-readable program instructions are executable by the processing unit for: driving the robot in surgical workflow according to the driving instructions.

19. The computer-assisted surgery system according to claim 1, further including a machine learning module and wherein the computer-readable program instructions are executable by the processing unit to train the machine learning module using the surgical procedure profile, the joint laxity data and the surgical plan.

20. The computer-assisted surgery system according to claim 19, further including outputting the machine learning module parametrized to generate surgical plans using surgical procedure profiles and joint laxity data.
